(19) United States
(12) Patent Application Publication
(10) Pub. No.: US 2019/0365553 A1
(43) Pub. Date: Dec. 5, 2019

(54) Spinal Specific Postural Correction Devices

(71) Applicant: Jason D. Drivere, New Wilmington, PA (US)

(72) Inventor: Jason D. Drivere, New Wilmington, PA (US)

(21) Appl. No.: 15/991,090

(22) Filed: May 29, 2018

(51) Int. Cl. A61F 5/01; A47G 9/10

(52) U.S. Cl. CPC A61F 5/01; A47G 2009/1018; A47G 9/1081

(57) ABSTRACT

Orthotic devices which utilize spinal traction and when properly placed helps impart the desired lordotic shape in the cervical spine, the thoracic spine, and the lumbar spine of a person are disclosed.

### BACKGROUND OF THE INVENTION

### 1. Field of the Disclosure

The present disclosure relates generally to orthotic devices for a spine of a person. More particularly, the present disclosure relates to orthotic devices for a cervical spine, a thoracic spine, and a lumbar spine of a person.

### 2. Description of the Related Art

Postural dysfunction or poor posture is caused when a person's spine is positioned in unnatural positions, in which the curves of the spine are emphasized and this results in the joints, muscles and vertebrae being in stressful positions. This prolonged poor positioning results in a buildup of pressure on these tissues. A great number of people suffer from the negative effects of bad posture.

Painful conditions associated with poor posture are very common and back pain is the most frequent complaint but research shows neck, shoulder and arm pain has become increasingly widespread as a result of postural dysfunction. Ligaments in the spine may become shortened and tight due to poor posture habits and/or previous injuries. These shortened ligaments and soft tissues can make it difficult to maintain good posture which may contribute to increased muscle tension, headaches, and neck pain.

### SUMMARY OF THE INVENTION

The present disclosure provides orthotic devices which utilize spinal traction and when properly placed helps impart the desired lordotic shape in the cervical spine, the thoracic spine, and the lumbar spine of a person.

The thoracic spine has a natural kyphosis or forward curvature. However, in cases of bad posture, it is increased more than normal so improving thoracic extension or improving spinal range of motion in extension is helpful to help offset the posture strain of the increased kyphosis. The orthotic devices of the present disclosure effectively stretch ligaments and other soft tissues and gently manipulate the spine to improve spinal intersegmental mobility and the ability to improve range of motion in spinal extension by improving the lordosis of the spine. The orthotic devices of the present disclosure also promote fluid and cellular exchange in and around the intervertebral discs. When properly applied to the spine by a person laying supine over the orthotic devices of the present disclosure, the orthotic devices supply a specific force forwardly in the sagittal plane to apply an angular/circular traction to the specified region of the spine, e.g., the cervical spine, the thoracic spine, and/or the lumbar spine of a person.

In accordance with an embodiment of the present invention, an orthotic device for a cervical spine and an upper thoracic spine of a person includes a base portion having a first base end and a second base end; a first protruding portion defining a first support surface configured to support the cervical spine at a first region, the first protruding portion spaced a first distance from the base portion; a second protruding portion defining a second support surface configured to support the cervical spine at a second region, the second protruding portion spaced a second distance from the base portion, wherein the second distance is less than the first distance; and a third protruding portion defining a third support surface configured to support the upper thoracic spine at a third region, the third protruding portion spaced a third distance from the base portion, wherein the third distance is less than the second distance.

In one configuration, the orthotic device tapers from the first base end to the second base end. In another configuration, the first support surface is a first convex surface. In yet another configuration, the second support surface is a second convex surface. In one configuration, the third support surface is a third convex surface. In another configuration, the orthotic device includes a fourth support surface between the first protruding portion and the second protruding portion. In yet another configuration, the orthotic device includes a fifth support surface between the second protruding portion and the third protruding portion. In one configuration, the fourth support surface is a first concave surface. In another configuration, the fifth support surface is a second concave surface. In yet another configuration, the first protruding portion is located at the first base end. In one configuration, the third protruding portion is located at the second base end. In another configuration, the orthotic device includes a ramp portion spanning the second base end and the third protruding portion. In yet another configuration, the second protruding portion is located between the first protruding portion and the third protruding portion. In one configuration, the second region is spaced from the first region.

In accordance with another embodiment of the present invention, an orthotic device for a thoracic spine of a person includes a base portion having a first base end and a second base end; a first protruding portion defining a first support surface configured to support the thoracic spine at a first region, the first protruding portion spaced a first distance from the base portion; and a second protruding portion defining a second support surface configured to support the thoracic spine at a second region, the second protruding portion spaced a second distance from the base portion, wherein the second distance is less than the first distance.

In one configuration, the first support surface is a first convex surface. In another configuration, the second support surface is a second convex surface. In yet another configuration, the first protruding portion is located at the second base end. In one configuration, the second protruding portion is located at the first base end. In another configuration, the orthotic device includes a first ramp portion spanning the second base end and the first protruding portion. In yet another configuration, the first ramp portion extends upwards from the second base end at an angle of 45 degrees. In one configuration, the orthotic device includes a second ramp portion spanning the first base end and the second protruding portion. In another configuration, the second ramp portion extends upwards from the first base end at an angle of 70 degrees.

In accordance with an embodiment of the present invention, an orthotic device for a lumbar spine of a person includes a base portion having a first base end and a second base end; a first protruding portion defining a first support surface configured to support the lumbar spine at a first region, the first protruding portion spaced a first distance from the base portion; a second protruding portion defining a second support surface configured to support the lumbar spine at a second region, the second protruding portion spaced a second distance from the base portion, wherein the second distance is less than the first distance; and a third protruding portion defining a third support surface configured to support the lumbar spine at a third region, the third protruding portion spaced a third distance from the base portion, wherein the third distance is less than the first distance, wherein the first protruding portion is located between the second protruding portion and the third protruding portion.

In one configuration, the first support surface is a first convex surface. In another configuration, the second support surface is a second convex surface. In yet another configuration, the third support surface is a third convex surface. In one configuration, the third protruding portion is located at the first base end. In another configuration, the second protruding portion is located at the second base end. In yet another configuration, the orthotic device includes a first ramp portion spanning the second base end and the second protruding portion. In one configuration, the first ramp portion extends upwards from the second base end at an angle of 60 degrees. In another configuration, the orthotic device includes a second ramp portion spanning the first base end and the third protruding portion. In yet another configuration, the second ramp portion extends upwards from the first base end at an angle of 45 degrees.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of three orthotic devices in accordance with an embodiment of the present invention.

FIG. 2 is a perspective view of an orthotic device for a cervical spine and an upper thoracic spine of a person in accordance with an embodiment of the present invention.

FIG. 3 is a first elevation view of an orthotic device for a cervical spine and an upper thoracic spine of a person in accordance with an embodiment of the present invention.

FIG. 4 is a second elevation view of an orthotic device for a cervical spine and an upper thoracic spine of a person in accordance with an embodiment of the present invention.

FIG. 5 is a third elevation view of an orthotic device for a cervical spine and an upper thoracic spine of a person in accordance with an embodiment of the present invention.

FIG. 6 is a fourth elevation view of an orthotic device for a cervical spine and an upper thoracic spine of a person in accordance with an embodiment of the present invention.

FIG. 7 is a fifth elevation view of an orthotic device for a cervical spine and an upper thoracic spine of a person in accordance with an embodiment of the present invention.

FIG. 8 is a sixth elevation view of an orthotic device for a cervical spine and an upper thoracic spine of a person in accordance with an embodiment of the present invention.

FIG. 9 is a perspective view of an orthotic device for a thoracic spine of a person in accordance with another embodiment of the present invention.

FIG. 10 is a first elevation view of an orthotic device for a thoracic spine of a person in accordance with another embodiment of the present invention.

FIG. 11 is a second elevation view of an orthotic device for a thoracic spine of a person in accordance with another embodiment of the present invention.

FIG. 12 is a third elevation view of an orthotic device for a thoracic spine of a person in accordance with another embodiment of the present invention.

FIG. 13 is a fourth elevation view of an orthotic device for a thoracic spine of a person in accordance with another embodiment of the present invention.

FIG. 14 is a fifth elevation view of an orthotic device for a thoracic spine of a person in accordance with another embodiment of the present invention.

FIG. 15 is a sixth elevation view of an orthotic device for a thoracic spine of a person in accordance with another embodiment of the present invention.

FIG. 16 is a perspective view of an orthotic device for a lumbar spine of a person in accordance with another embodiment of the present invention.

FIG. 17 is a first elevation view of an orthotic device for a lumbar spine of a person in accordance with another embodiment of the present invention.

FIG. 18 is a second elevation view of an orthotic device for a lumbar spine of a person in accordance with another embodiment of the present invention.

FIG. 19 is a third elevation view of an orthotic device for a lumbar spine of a person in accordance with another embodiment of the present invention.

FIG. 20 is a fourth elevation view of an orthotic device for a lumbar spine of a person in accordance with another embodiment of the present invention.

FIG. 21 is a fifth elevation view of an orthotic device for a lumbar spine of a person in accordance with another embodiment of the present invention.

FIG. 22 is a sixth elevation view of an orthotic device for a lumbar spine of a person in accordance with another embodiment of the present invention.

FIG. 23 is a perspective view of an orthotic device for a cervical spine and an upper thoracic spine in use by a person in accordance with an embodiment of the present invention.

FIG. 24 is a perspective view of an orthotic device for a thoracic spine in use by a person in accordance with another embodiment of the present invention.

FIG. 25 is a perspective view of an orthotic device for a lumbar spine in use by a person in accordance with another embodiment of the present invention.

FIG. 26 is a perspective view of a person showing an example of a normal lordosis or backward curvature of the spine in accordance with an embodiment of the present invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

The present disclosure provides orthotic devices which utilize spinal traction and when properly placed helps impart the desired lordotic shape in the cervical spine, the thoracic spine, and the lumbar spine of a person.

The thoracic spine has a natural kyphosis or forward curvature. However, in cases of bad posture, it is increased more than normal so improving thoracic extension or improving spinal range of motion in extension is helpful to help offset the posture strain of the increased kyphosis. The orthotic devices of the present disclosure effectively stretch ligaments and other soft tissues and gently manipulate the spine to improve spinal intersegmental mobility and the ability to improve range of motion in spinal extension by improving the lordosis of the spine. The orthotic devices of the present disclosure also promote fluid and cellular exchange in and around the intervertebral discs. When properly applied to the spine by a person laying supine over the orthotic devices of the present disclosure, the orthotic devices supply a specific force forwardly in the sagittal plane to apply an angular/circular traction to the specified region of the spine, e.g., the cervical spine, the thoracic spine, and/or the lumbar spine of a person.

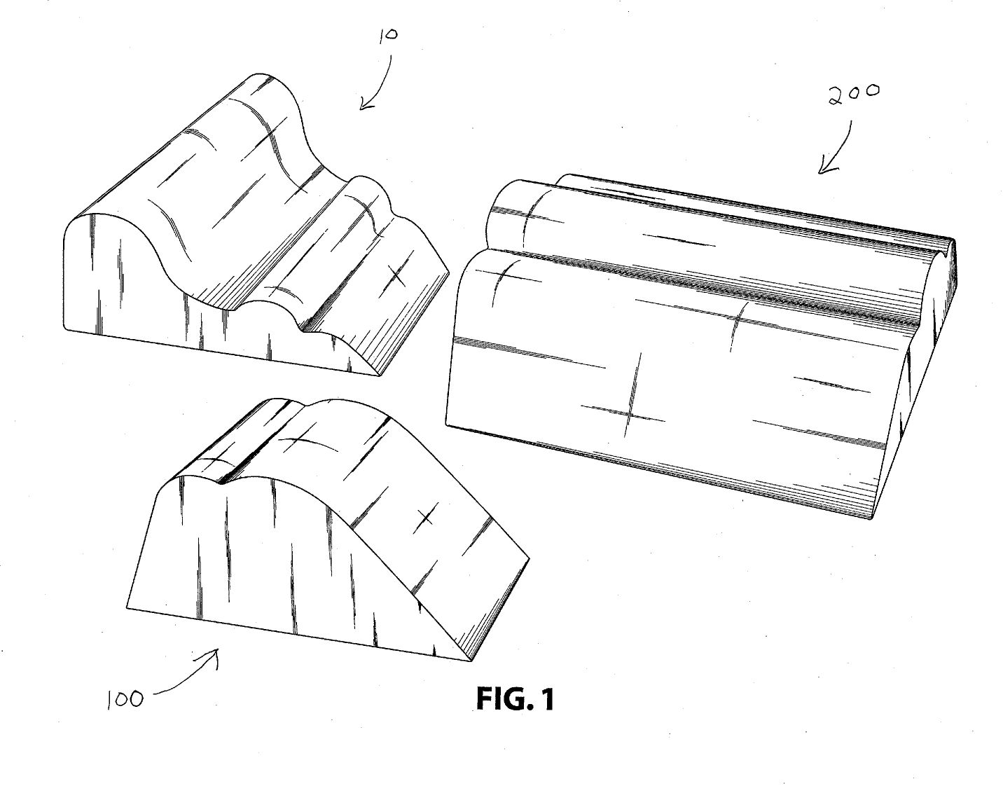

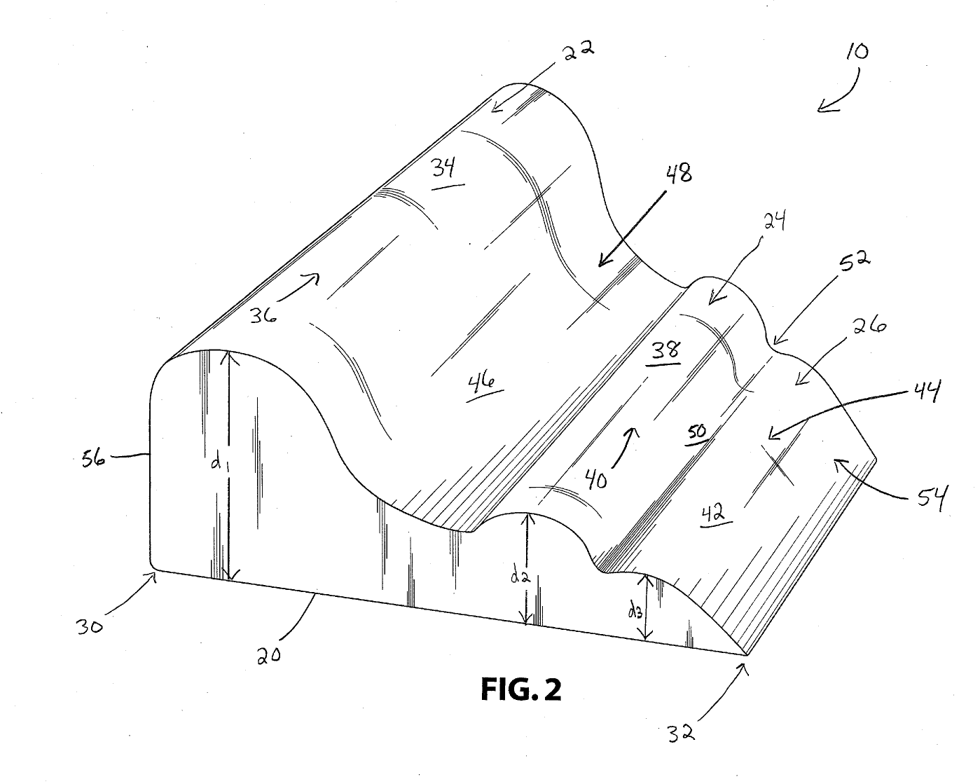

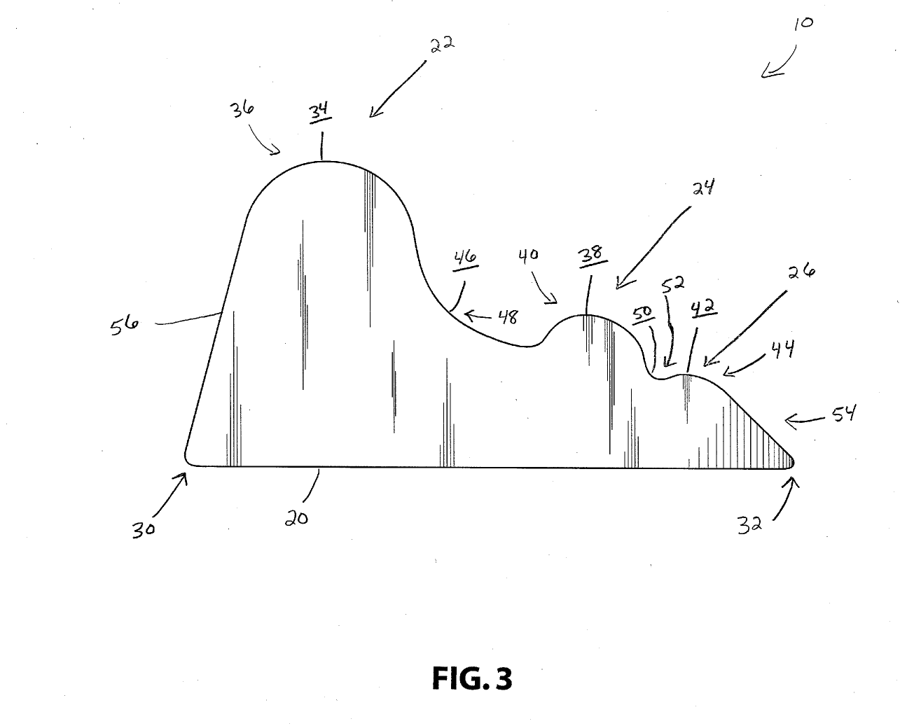

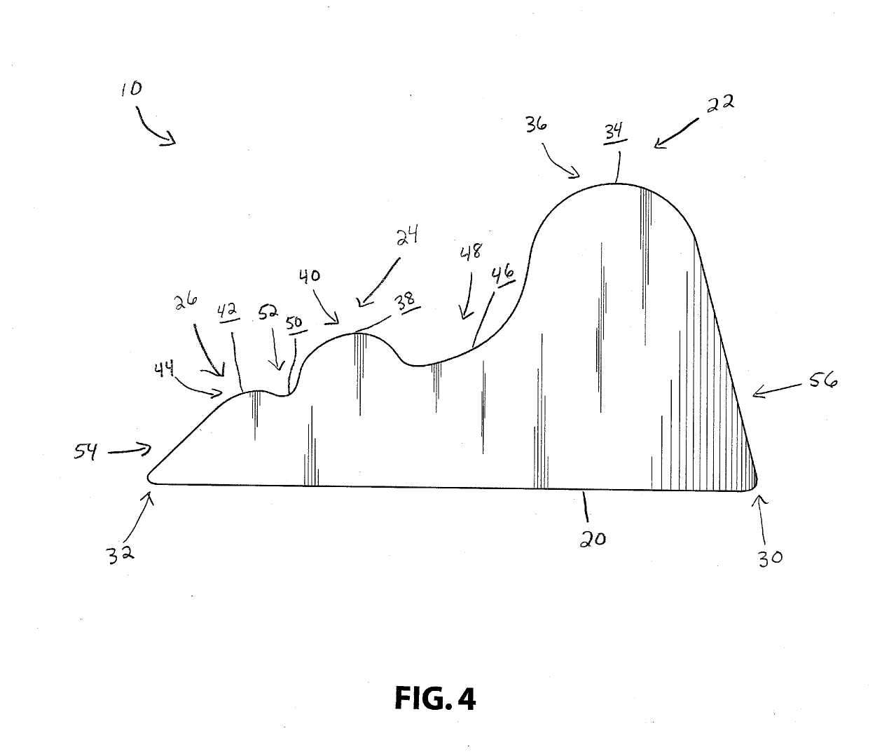

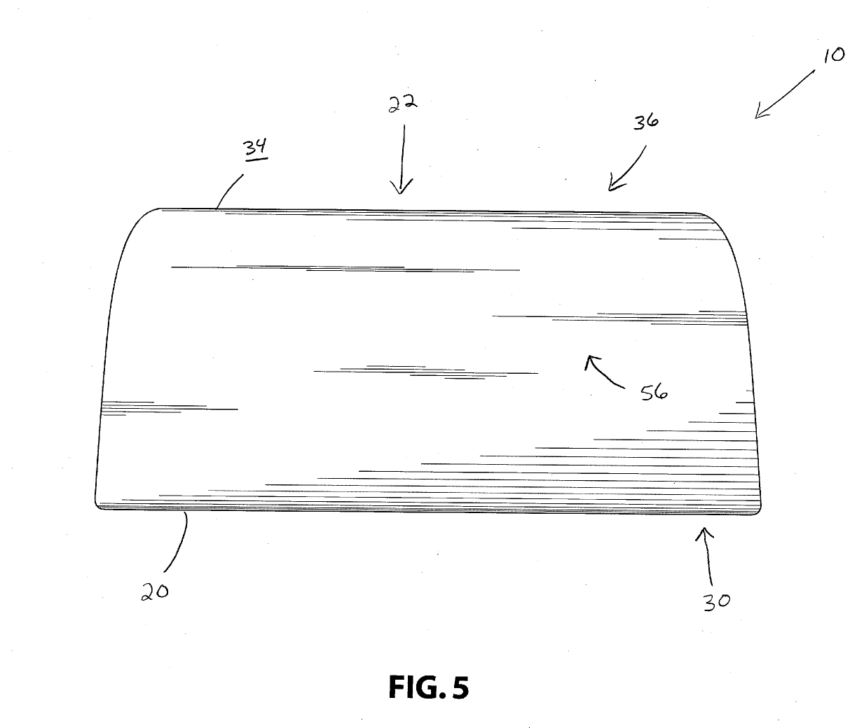

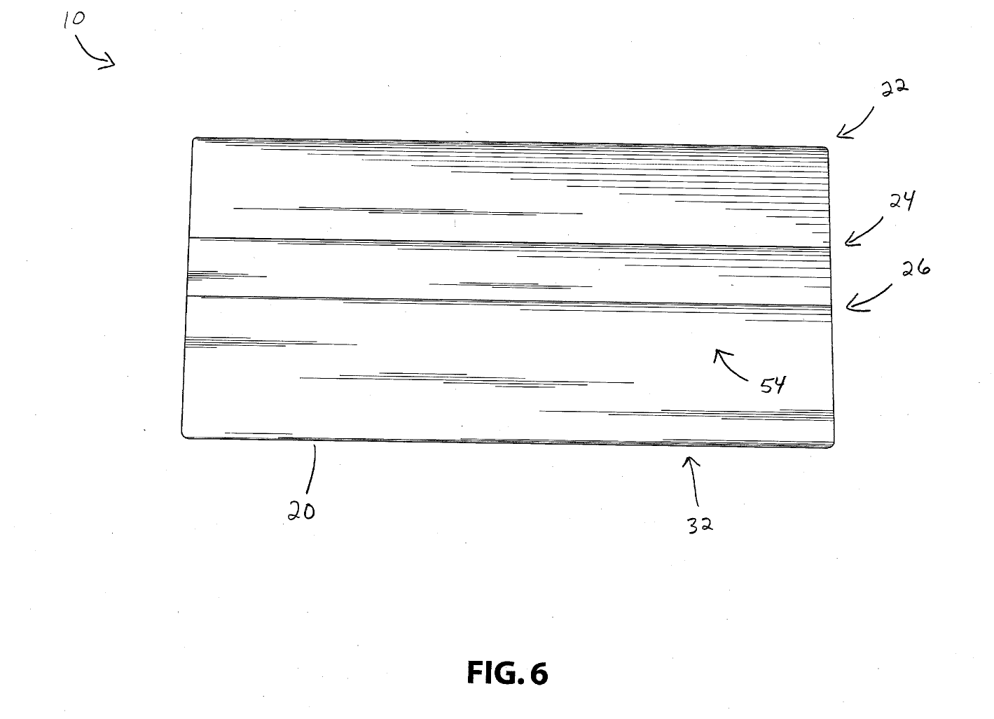

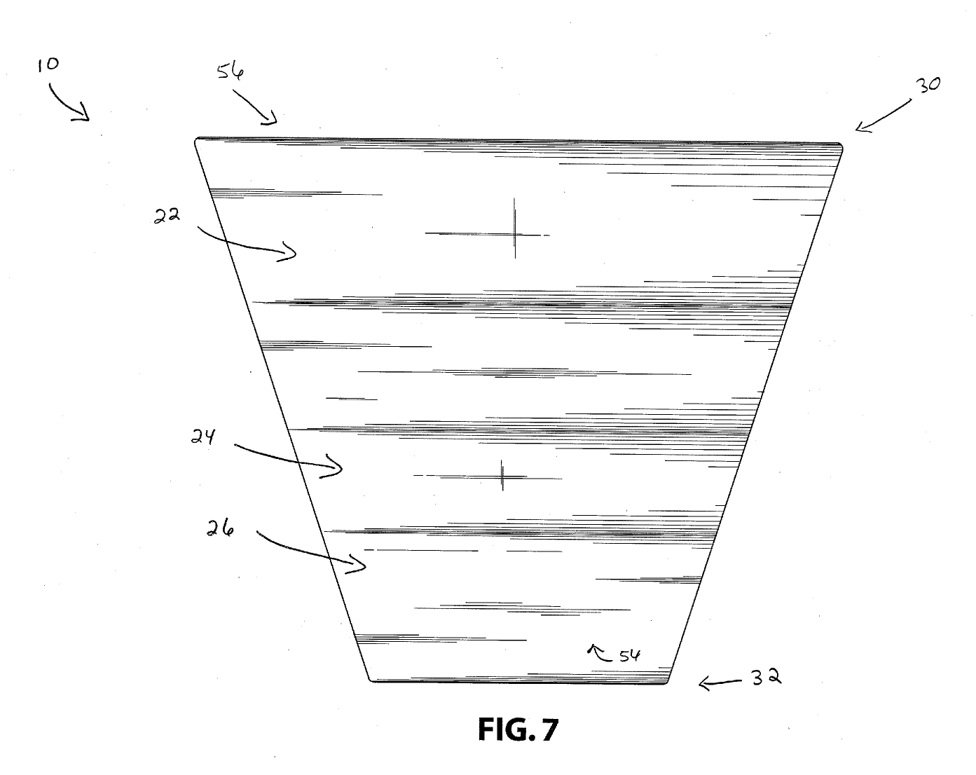

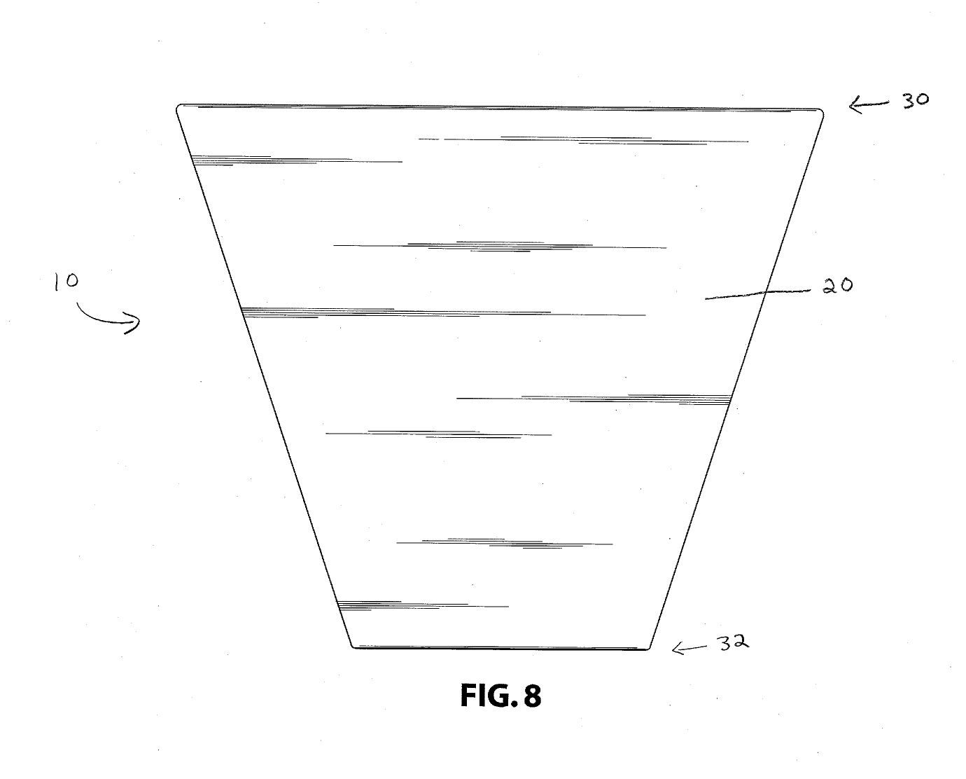

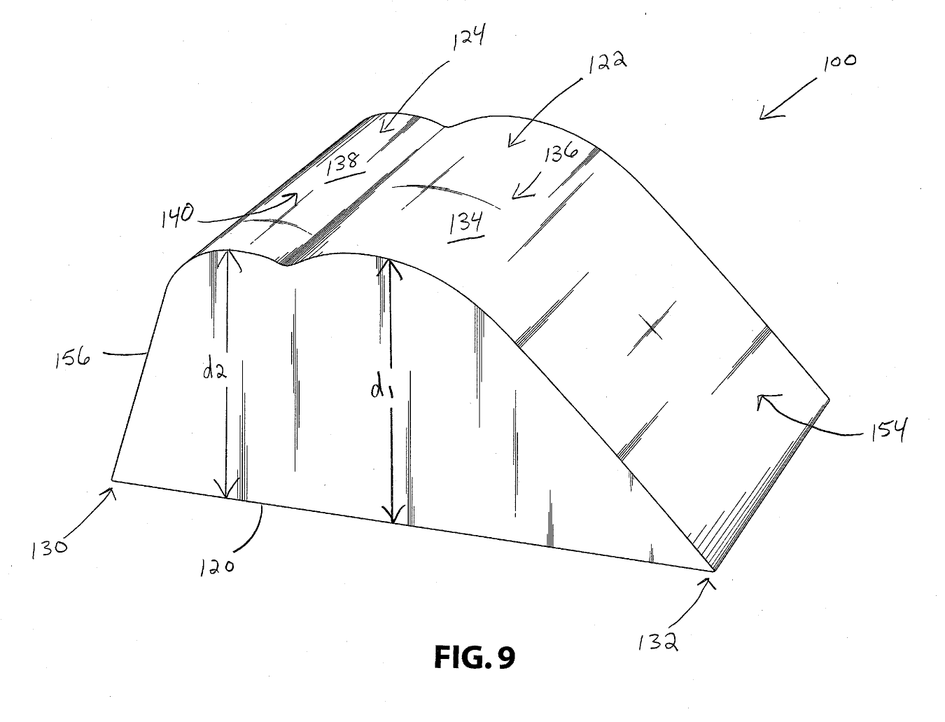

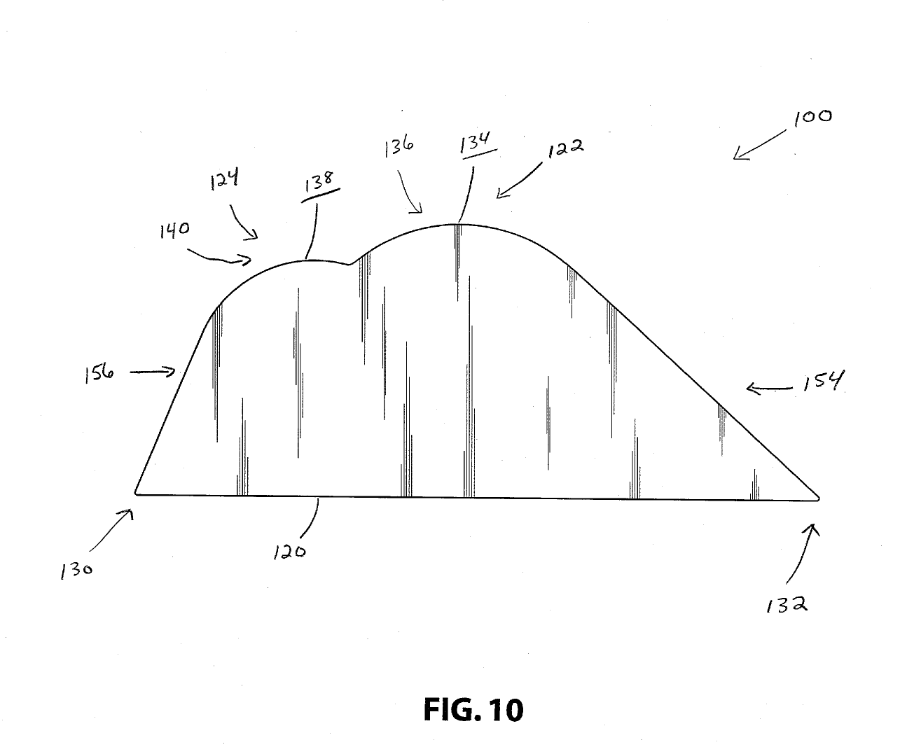

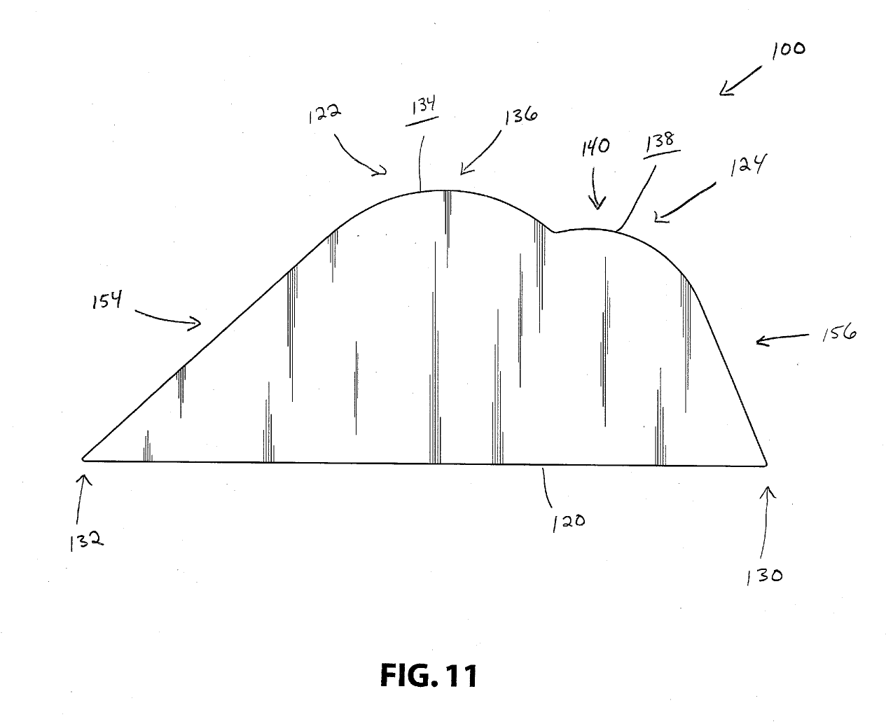

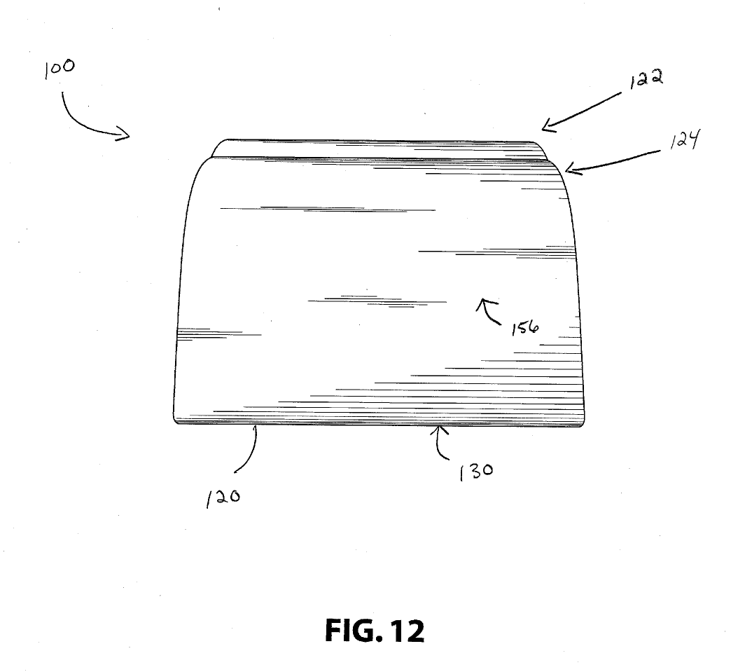

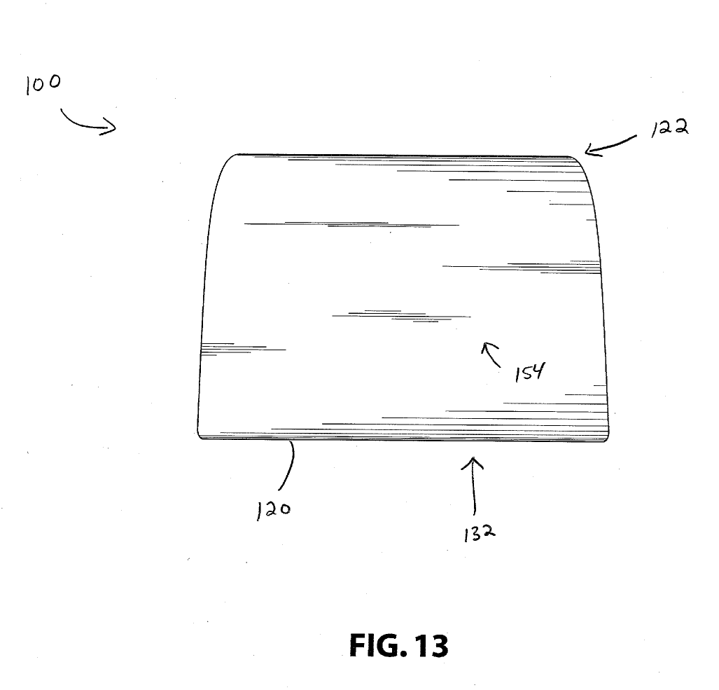

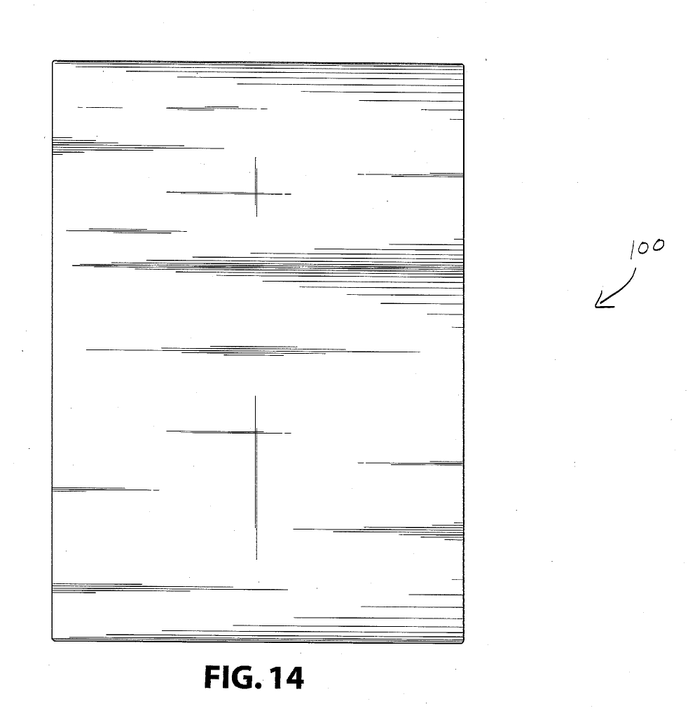

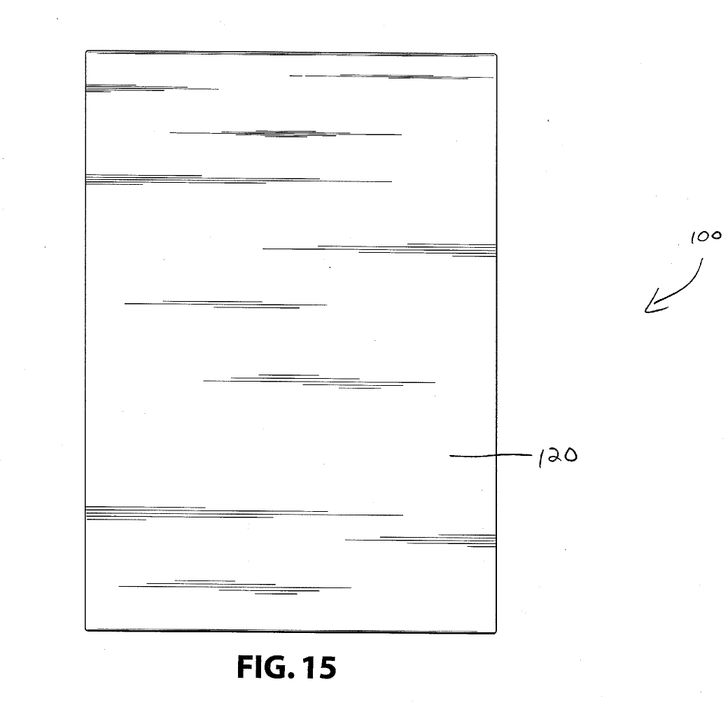

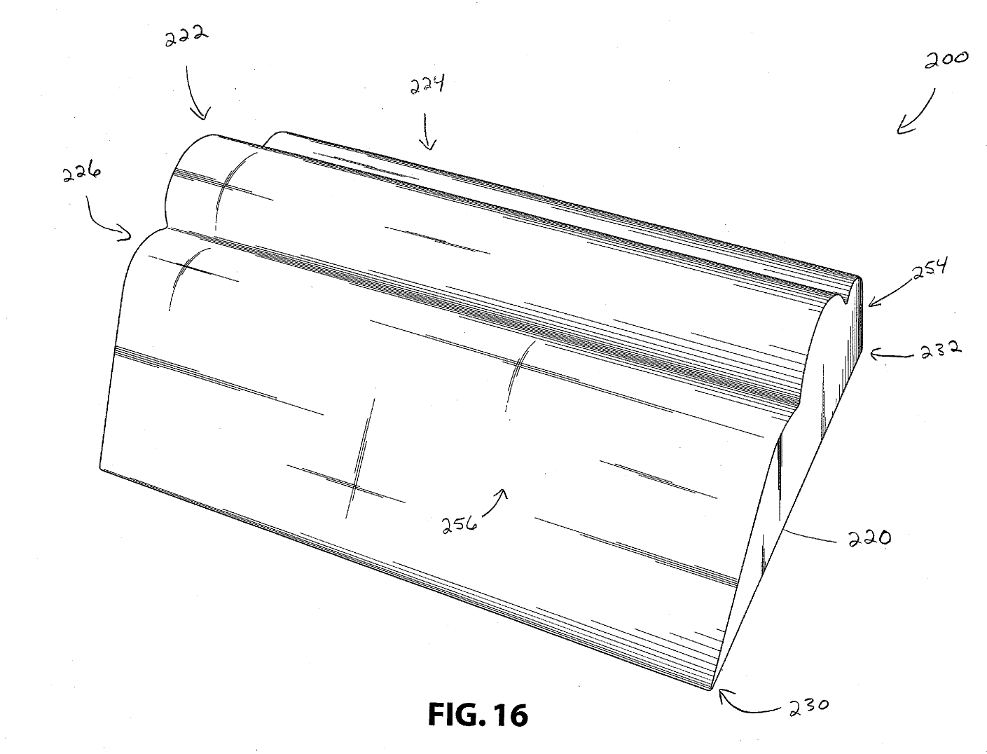

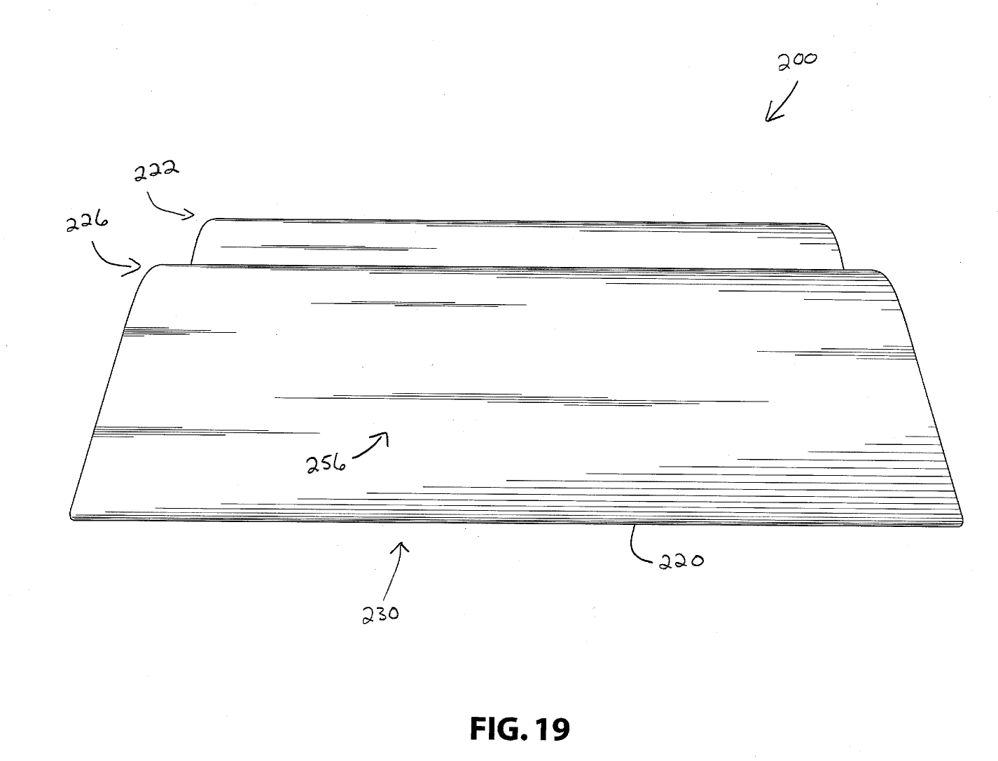

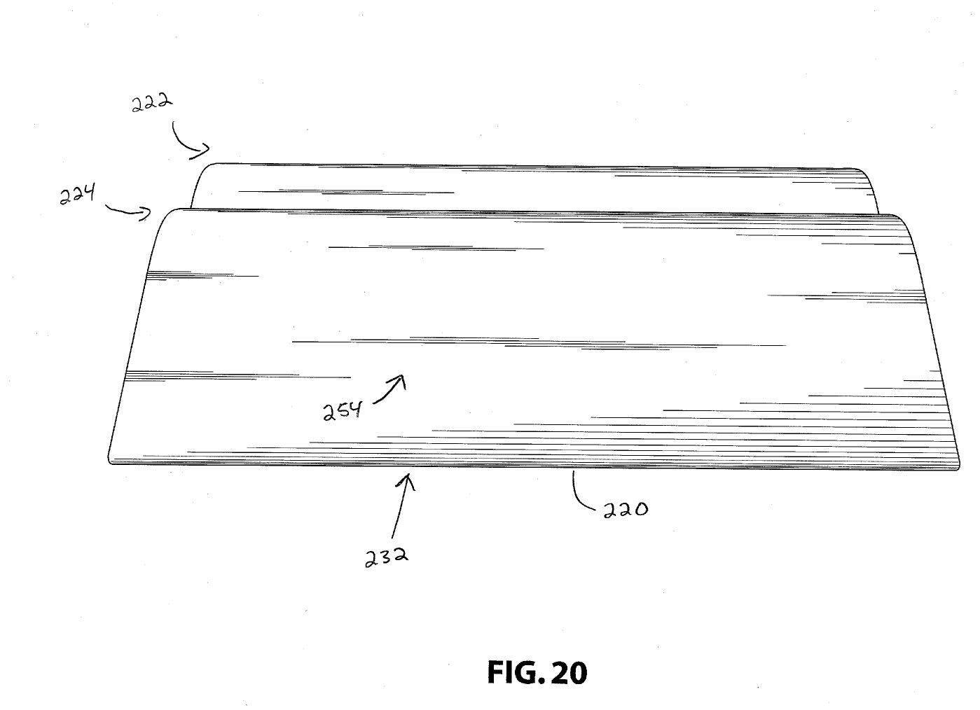

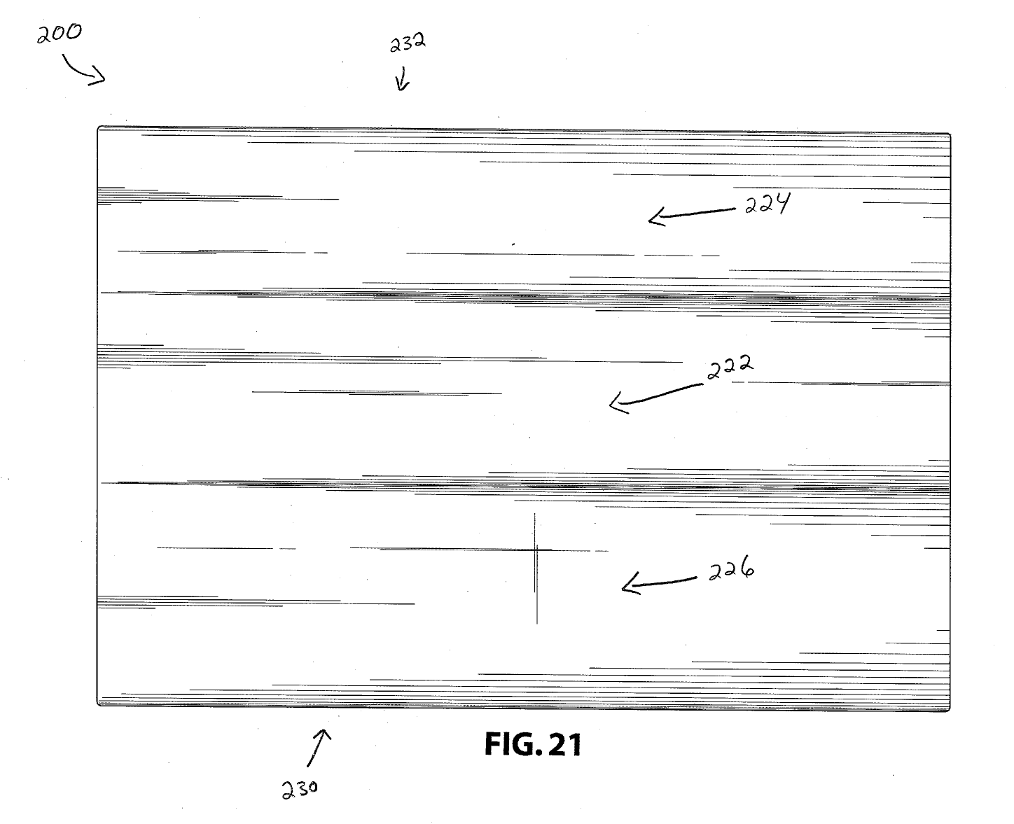

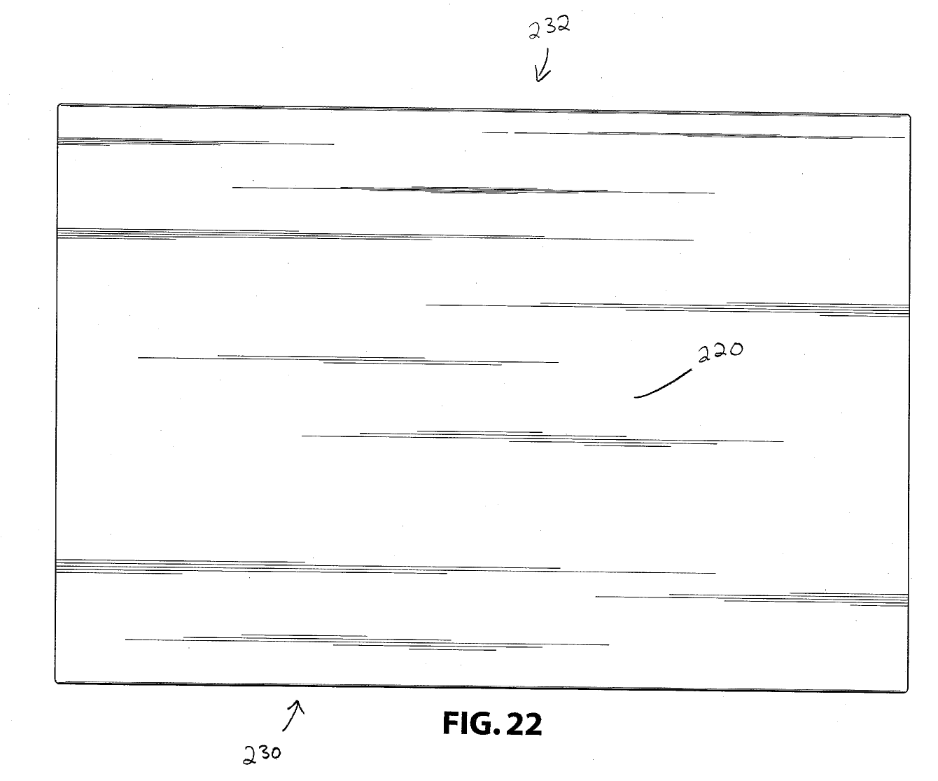

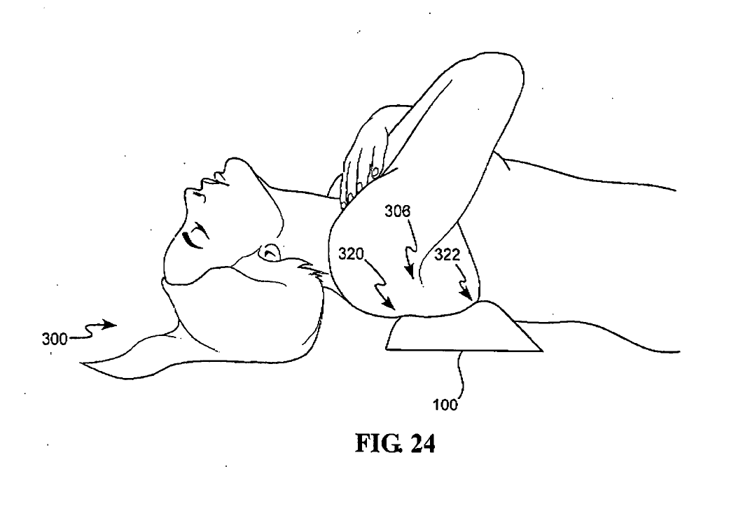

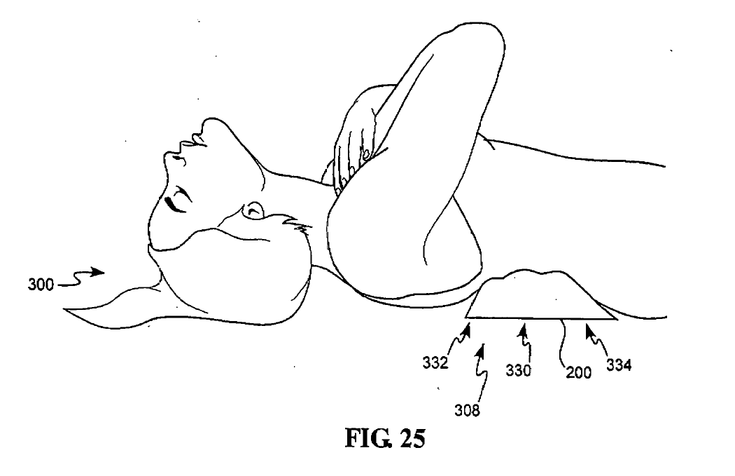

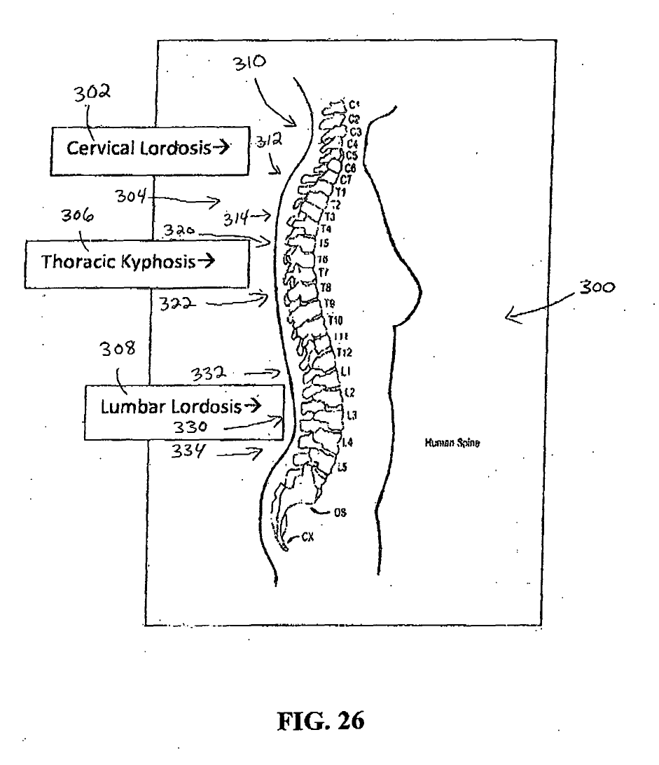

FIG. 1 illustrates an exemplary embodiment of orthotic devices of the present disclosure. Referring to FIGS. 2-8 and 23, an orthotic device 10 for a cervical spine 302 and an upper thoracic spine 304 of a person 300 is illustrated. Referring to FIGS. 9-15 and 24, an orthotic device 100 for a thoracic spine 306 of a person 300 is illustrated. Referring to FIGS. 16-22 and 25, an orthotic device 200 for a lumbar spine 308 of a person 300 is illustrated. Referring to FIG. 26, different regions of a cervical spine 302, an upper thoracic spine 304, a thoracic spine 306, and a lumbar spine 308 of a person 300 is illustrated.

Referring to FIGS. 2-8, an orthotic device 10 for a cervical spine 302 and an upper thoracic spine 304 of a person 300 generally includes a base portion 20, a first protruding portion 22, a second protruding portion 24, and a third protruding portion 26. The base portion 20 has a first base end 30 and a second base end 32.

Referring to FIGS. 2-8, in one embodiment, the first protruding portion 22 defines a first support surface 34 that is configured to support the cervical spine 302 at a first region 310. The first protruding portion 22 is spaced a first distance d1 from the base portion 20. In one embodiment, the first protruding portion 22 is spaced a first distance d1 of 3.5 inches from the base portion 20. In other embodiments, the first protruding portion 22 may be spaced other distances from the base portion 20 for a desired application and/or person depending on their size.

In one embodiment, the first support surface 34 is a first convex surface 36. Referring to FIGS. 2-8, the first protruding portion 22 is located at the first base end 30. In one embodiment, the first protruding portion 22 has a diameter of approximately two (2) inches. In other embodiments, the first protruding portion 22 may have other diameters for a desired application and/or person depending on their size.

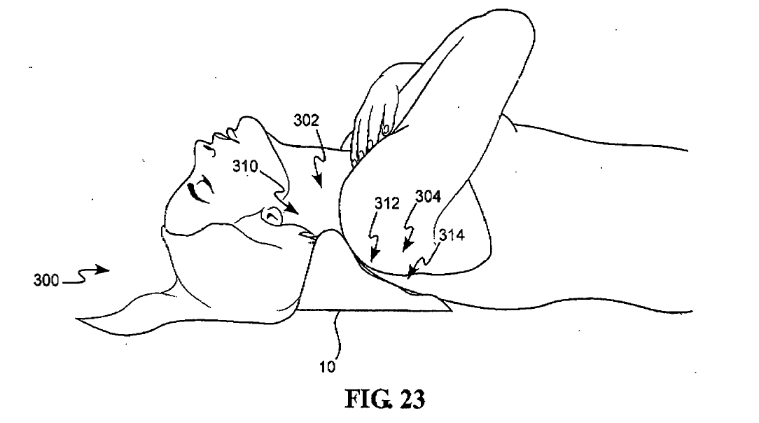

Referring to FIGS. 2-8, in one embodiment, the second protruding portion 24 defines a second support surface 38 that is configured to support the cervical spine 302 at a second region 312. Referring to FIGS. 23 and 26, the second region 312 of the cervical spine 302 is spaced from the first region 310 of the cervical spine 302.

Advantageously, in this manner, the orthotic device 10 of the present disclosure supports the cervical spine 302 at two separate regions, i.e., the first region 310 and the second region 312. A device that only supports the cervical spine at one region will cause extension of the mid cervical spine but also an increased forward flexion of the upper thoracic spine. Such a device that only supports the cervical spine at one region is not advantageous when trying to improve extension in the cervical spine because an increased kyphosis at the upper thoracic spine and cervicothoracic region can contribute to a forward head posture. Furthermore, the orthotic device 10 of the present disclosure supporting the cervical spine 302 at two separate regions also provides a more comfortable device for a person 300 and provides a more effective extension stretch to the cervical spine 302. Also, the orthotic device 10 of the present disclosure helps keep the cervicothoracic region and upper thoracic region in a more neutral position which allows for a more comfortable device for a person 300 and provides a more effective extension stretch to the cervical spine 302.

Referring to FIGS. 2-8, in one embodiment, the second protruding portion 24 is spaced a second distance d2 from the base portion 20. In one embodiment, the second distance d2 is less than the first distance d1. In one embodiment, the second protruding portion 24 is spaced a second distance d2 of approximately 1.5 to 2.0 inches from the base portion 20. In other embodiments, the second protruding portion 24 may be spaced other distances from the base portion 20 for a desired application and/or person depending on their size.

In one embodiment, the second support surface 38 is a second convex surface 40. Referring to FIGS. 2-8, the second protruding portion 24 is located between the first protruding portion 22 and the third protruding portion 26. In one embodiment, the second protruding portion 24 has a diameter of approximately 1.5 inches. In other embodiments, the second protruding portion 24 may have other diameters for a desired application and/or person depending on their size.

Referring to FIGS. 2-8, in one embodiment, the third protruding portion 26 defines a third support surface 42 that is configured to support the upper thoracic spine 304 at a third region 314.

Advantageously, in this manner, the orthotic device 10 of the present disclosure supports the spine 301 of a person 300 at three separate points of contact. In one embodiment, the orthotic device 10 of the present disclosure supports the spine 301 of a person 300 at three separate convex points of contact. Furthermore, the orthotic device 10 of the present disclosure supporting the cervical spine 302 at two separate regions and the upper thoracic spine 304 at a third region 314 also provides a more comfortable device for a person 300 and provides a more effective extension stretch to the cervical spine 302. The orthotic device 10 of the present disclosure supporting the spine 301 of a person 300 at three separate points of contact provides an enhanced orthotic device advantageous over conventional devices. Also, the orthotic device 10 of the present disclosure helps keep the cervicothoracic region and upper thoracic region in a more neutral position which allows for a more comfortable device for a person 300 and provides a more effective extension stretch to the cervical spine 302.

In one embodiment, the third protruding portion 26 is spaced a third distance d3 from the base portion 20. In one embodiment, the third distance d3 is less than the second distance d2. In one embodiment, the third support surface 42 is a third convex surface 44. In one embodiment, the third protruding portion 26 is located at the second base end 32. In one embodiment, the third protruding portion 26 has a diameter of approximately one (1) inch. In other embodiments, the third protruding portion 26 may have other diameters for a desired application and/or person depending on their size.

In one embodiment, the third protruding portion 26 is spaced a third distance d3 of approximately 1.0 to 1.5 inches from the base portion 20. In other embodiments, the third protruding portion 26 may be spaced other distances from the base portion 20 for a desired application and/or person depending on their size.

Referring to FIGS. 2-8, in one embodiment, an orthotic device 10 for a cervical spine 302 and an upper thoracic spine 304 of a person 300 also includes a fourth support surface 46 and a fifth support surface 50. In one embodiment, the fourth support surface 46 is located between the first protruding portion 22 and the second protruding portion 24. In one embodiment, the fourth support surface 46 is a first concave surface 48. The fifth support surface 50 is located between the second protruding portion 24 and the third protruding portion 26. In one embodiment, the fifth support surface 50 is a second concave surface 52.

Referring to FIGS. 2-8, in one embodiment, an orthotic device 10 for a cervical spine 302 and an upper thoracic spine 304 of a person 300 also includes a first ramp portion 54 that spans the second base end 32 and the third protruding portion 26.

In some embodiments, the orthotic device 10 for a cervical spine 302 and an upper thoracic spine 304 of a person 300 may also include a second ramp portion 56 that spans the first base end 30 and the first protruding portion 22.

Referring to FIGS. 2, 7, and 8, in one embodiment, the orthotic device 10 tapers from the first base end 30 to the second base end 32. This taper helps accommodate the space between a person's shoulder blades in an efficient and comfortable manner. Advantageously, in this manner, the orthotic device 10 of the present disclosure helps keep the cervicothoracic region and upper thoracic region in a more neutral position which allows for a more comfortable device for a person 300 and provides a more effective extension stretch to the cervical spine 302. The orthotic device 10 of the present disclosure supporting the spine 301 of a person 300 at three separate points of contact and having the above-described taper provides an enhanced orthotic device advantageous over conventional devices.

In one embodiment, the orthotic device 10 has an eight (8) inch width at the first base end 30 and a four (4) inch width at the second base end 32. The base portion 20 tapers from the first base end 30 to the second base end 32. In other embodiments, the orthotic device 10 may have other widths at the first base end 30 and other widths at the second base end 32 for a desired application and/or person depending on their size.

In one embodiment, the orthotic device 10 of the present disclosure is made of a four (4) pound cross-linked polyethylene foam. In one embodiment, the orthotic device 10 of the present disclosure is approximately 7.5 inches in length.

During use of the orthotic device 10 of the present disclosure, a person lays in a supine position over the orthotic device 10 such that the orthotic device 10 supplies a specific force forwardly in the sagittal plane to apply an angular/circular traction to the cervical spine 302 at two separate regions, i.e., the first region 310 and the second region 312, and an upper thoracic spine 304 of a person 300.

Referring to FIGS. 9-15, an orthotic device 100 for a thoracic spine 306 of a person 300 generally includes a base portion 120, a first protruding portion 122, and a second protruding portion 124. The base portion 120 has a first base end 130 and a second base end 132.

Referring to FIGS. 9-15, in one embodiment, a first protruding portion 122 defines a first support surface 134 that is configured to support the thoracic spine 306 at a first region 320. The first protruding portion 122 is spaced a first distance d1 from the base portion 120. In one embodiment, the first protruding portion 122 is spaced a first distance d1 of 3.5 inches from the base portion 120. In other embodiments, the first protruding portion 122 may be spaced other distances from the base portion 120 for a desired application and/or person depending on their size.

In one embodiment, the first support surface 134 is a first convex surface 136. Referring to FIGS. 9-15, the first protruding portion 122 is located at the second base end 132. In one embodiment, the first protruding portion 122 has a diameter of approximately three (3) inches. In other embodiments, the first protruding portion 122 may have other diameters for a desired application and/or person depending on their size.

Advantageously, in this manner, the first protruding portion 122 of the orthotic device 100 of the present disclosure supports and provides a circular traction stretch to a person's thoracic spine into extension.

Referring to FIGS. 9-15, in one embodiment, the second protruding portion 124 defines a second support surface 138 that is configured to support the thoracic spine 306 at a second region 322. The second protruding portion 124 is spaced a second distance d2 from the base portion 120. In one embodiment, the second distance d2 is less than the first distance d1. In one embodiment, the second protruding portion 124 is spaced a second distance d2 of approximately 2.5 to 3.0 inches from the base portion 120. In other embodiments, the second protruding portion 124 may be spaced other distances from the base portion 120 for a desired application and/or person depending on their size.

In one embodiment, the second support surface 138 is a second convex surface 140. In one embodiment, the second protruding portion 124 is located at the first base end 130. In one embodiment, the second protruding portion 124 has a diameter of approximately two (2) inches. In other embodiments, the second protruding portion 124 may have other diameters for a desired application and/or person depending on their size.

Advantageously, in this manner, the second protruding portion 124 of the orthotic device 100 of the present disclosure provides a second support point that distributes a person's body weight over two support points, i.e., the first protruding portion 122 and the second protruding portion 124, which thereby decreases the force of the stretch of the thoracic region. In this manner, the orthotic device 100 of the present disclosure supporting the thoracic spine 306 at two separate regions also provides a more comfortable device for a person 300 and provides a more effective extension stretch to the thoracic spine 306.

Referring to FIGS. 9-15, an orthotic device 100 for a thoracic spine 306 of a person 300 also includes a first ramp portion 154 spanning the second base end 132 and the first protruding portion 122. In one embodiment, the first ramp portion 154 extends upwards from the second base end 132 at an angle of 45 degrees.

Referring to FIGS. 9-15, an orthotic device 100 for a thoracic spine 306 of a person 300 also includes a second ramp portion 156 spanning the first base end 130 and the second protruding portion 124. In one embodiment, the second ramp portion 156 extends upwards from the first base end 130 at an angle of 70 degrees.

In one embodiment, the orthotic device 100 of the present disclosure is made of a four (4) pound cross-linked polyethylene foam. In one embodiment, the orthotic device 100 of the present disclosure is approximately 8.5 inches in length. In one embodiment, the orthotic device 100 of the present disclosure is approximately 4 inches in width.

During use of the orthotic device 100 of the present disclosure, a person lays in a supine position over the orthotic device 100 such that the orthotic device 100 supplies a specific force forwardly in the sagittal plane to apply an angular/circular traction to the thoracic spine 306, at two separate regions, of a person 300.

Referring to FIGS. 16-22, an orthotic device 200 for a lumbar spine 308 of a person 300 generally includes a base portion 220, a first protruding portion 222, a second protruding portion 224, and a third protruding portion 226. The base portion 220 has a first base end 230 and a second base end 232.

Referring to FIGS. 16-22, in one embodiment, the first protruding portion 222 defines a first support surface 234 that is configured to support the lumbar spine 308 at a first region 330. The first protruding portion 222 is spaced a first distance d1 from the base portion 220. In one embodiment, the first protruding portion 222 is spaced a first distance d1 of approximately 3.5 to 4.0 inches from the base portion 220. In other embodiments, the first protruding portion 222 may be spaced other distances from the base portion 220 for a desired application and/or person depending on their size.

In one embodiment, the first support surface 234 is a first convex surface 236. In one embodiment, the first protruding portion 222 is located between the second protruding portion 224 and the third protruding portion 226. In one embodiment, the first protruding portion 222 has a diameter of approximately two (2) inches. In other embodiments, the first protruding portion 222 may have other diameters for a desired application and/or person depending on their size.

Advantageously, in this manner, the first protruding portion 222 of the orthotic device 200 of the present disclosure supports the lumbar spine 308 and provides a circular traction stretch which extends the lumbar spine into a lordosis.

Referring to FIGS. 16-22, in one embodiment, the second protruding portion 224 defines a second support surface 238 that is configured to support the lumbar spine 308 at a second region 332. In one embodiment, the second protruding portion 224 is spaced a second distance d2 from the base portion 220. In one embodiment, the second distance d2 is less than the first distance d1. In one embodiment, the second protruding portion 224 is spaced a second distance d2 of approximately 3.0 inches from the base portion 220. In other embodiments, the second protruding portion 224 may be spaced other distances from the base portion 220 for a desired application and/or person depending on their size.

In one embodiment, the second support surface 238 is a second convex surface 240. In one embodiment, the second protruding portion 224 is located at the second base end 232. In one embodiment, the second protruding portion 224 has a diameter of approximately 1.5 inches. In other embodiments, the second protruding portion 224 may have other diameters for a desired application and/or person depending on their size.

Referring to FIGS. 16-22, in one embodiment, the third protruding portion 226 defines a third support surface 242 that is configured to support the lumbar spine 308 at a third region 334.

Advantageously, in this manner, the orthotic device 200 of the present disclosure supports the lumbar spine 308 of a person 300 at three separate points of contact, i.e., at the first protruding portion 222, the second protruding portion 224, and the third protruding portion 226. Furthermore, the three supporting portions 222, 224, 226 together act to distribute the force and the weight of a person 300 over three points when lying over the orthotic device 200. In this manner, the orthotic device 200 of the present disclosure supporting the lumbar spine 308 at three separate regions provides a more comfortable device for a person 300 and provides a more effective extension stretch to the lumbar spine 308. The design of the orthotic device 200 also allows the device 200 to be used as a seated lumbar support.

Advantageously, by having the first protruding portion 222 being the tallest supporting surface and being centrally located allows the orthotic device 200 to provide more stability and less sliding and shifting of the orthotic device 200 when a person's body weight is pressed down upon the orthotic device 200. Furthermore, by having the first protruding portion 222 being the tallest supporting surface and being centrally located allows the orthotic device 200 to be used as a seated lumbar support since the orthotic device 200 provides more stability and less sliding and shifting of the orthotic device 200.

In one embodiment, the third protruding portion 226 is spaced a third distance d3 from the base portion 220. In one embodiment, the third distance d3 is less than the first distance d1. In one embodiment, the third protruding portion 226 is spaced a third distance d3 of approximately 3.0 inches from the base portion 220. In other embodiments, the third protruding portion 226 may be spaced other distances from the base portion 220 for a desired application and/or person depending on their size.

In one embodiment, the third support surface 242 is a third convex surface 244. In one embodiment, the third protruding portion 226 is located at the first base end 230. In one embodiment, the third protruding portion 226 has a diameter of approximately two (2) inches. In other embodiments, the third protruding portion 226 may have other diameters for a desired application and/or person depending on their size.

Referring to FIGS. 16-22, in one embodiment, the orthotic device 200 also includes a first ramp portion 254 that spans the second base end 232 and the second protruding portion 224. In one embodiment, the first ramp portion 254 extends upwards from the second base end 232 at an angle of 60 degrees.

Referring to FIGS. 16-22, in one embodiment, the orthotic device 200 also includes a second ramp portion 256 that spans the first base end 230 and the third protruding portion 226. In one embodiment, the second ramp portion 256 extends upwards from the first base end 230 at an angle of 45 degrees.

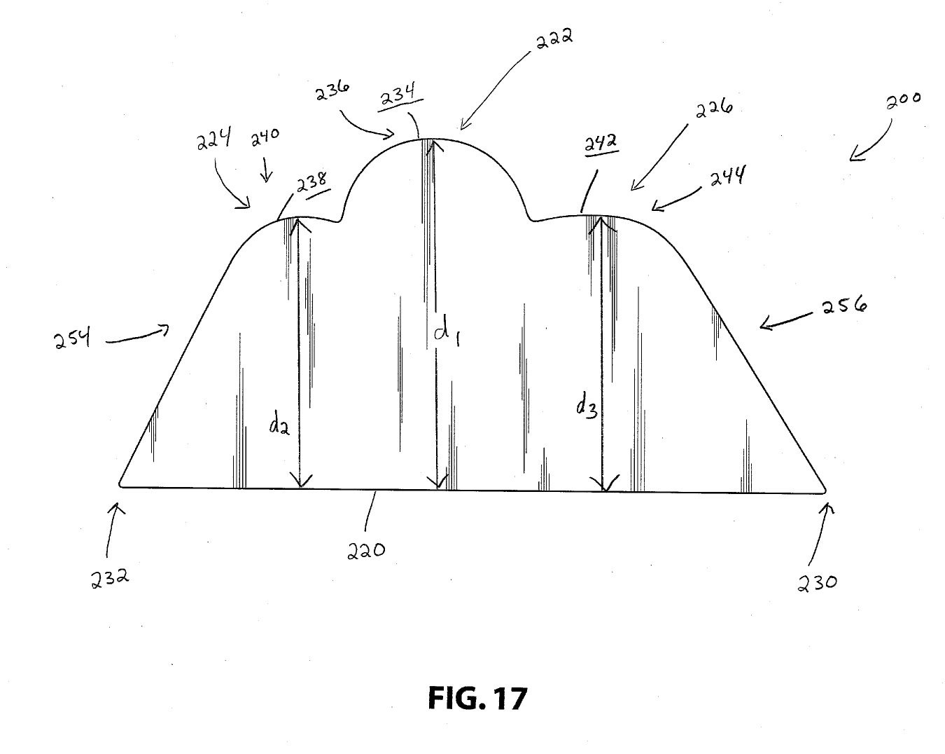

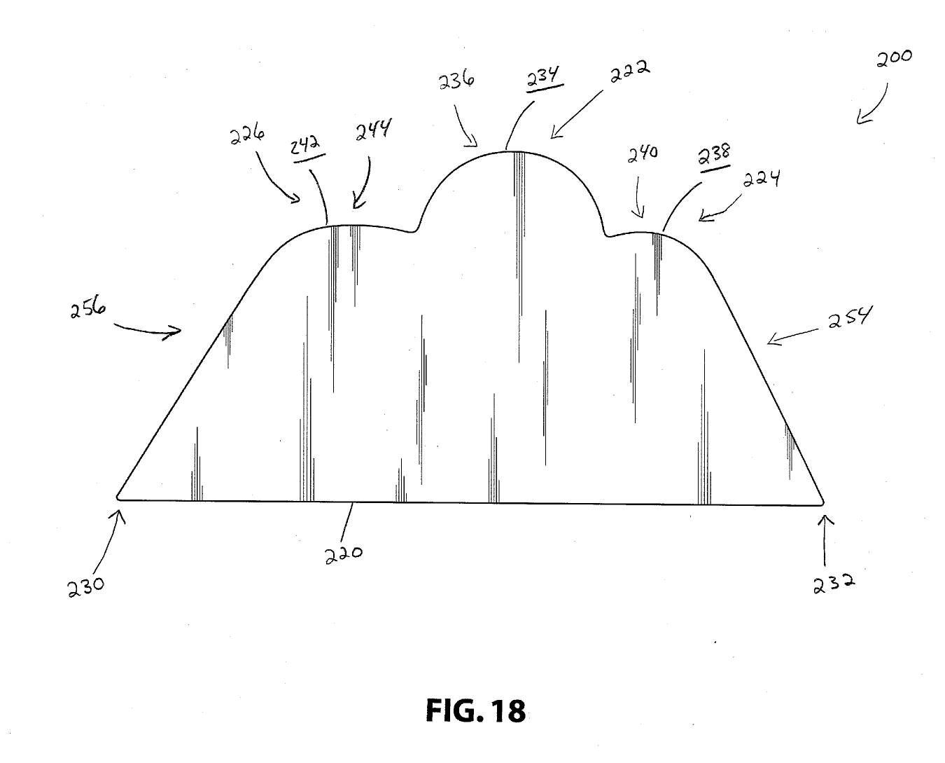

In one embodiment, referring to FIG. 17, the second protruding portion 224 is located approximately two (2) inches from the second base end 232 and the third protruding portion 226 is located approximately 2.3 to 2.5 inches from the first base end 230. Advantageously, this allows the orthotic device 200 of the present disclosure to be reversible, i.e., the orthotic device 200 can be rotated between two positions. For example, in a first configuration, the orthotic device 200 can be positioned as shown in FIG. 17, with the first base end 230 pointing downwards, e.g., towards the feet of a person. In this manner, the orthotic device 200 is able to more efficiently accommodate a taller person who will have a longer lumbar spine 308. Furthermore, in a second configuration, the orthotic device 200 can be positioned as shown in FIG. 18, with the second base end 232 pointing downwards, e.g., towards the feet of a person. In this manner, the orthotic device 200 is able to more efficiently accommodate a shorter person who will have a shorter lumbar spine 308. Advantageously, the orthotic device 200 of the present disclosure allows for customization and reversibility of the orthotic device 200 to accommodate people based on their height.

In one embodiment, the orthotic device 200 of the present disclosure is made of a two (2) pound cross-linked polyethylene foam. In one embodiment, the orthotic device 200 of the present disclosure is approximately 7.5 to 8.0 inches in length. In one embodiment, the orthotic device 200 of the present disclosure is approximately 10.0 inches in width.

During use of the orthotic device 200 of the present disclosure, a person lays in a supine position over the orthotic device 200 such that the orthotic device 200 supplies a specific force forwardly in the sagittal plane to apply an angular/circular traction to the lumbar spine 308 of a person 300.

In some embodiments, an orthotic device of the present disclosure can also be used in a sitting position to help improve posture.

The present disclosure provides orthotic devices which utilize spinal traction and when properly placed helps impart the desired lordotic shape in the cervical spine, the thoracic spine, and the lumbar spine of a person.

The thoracic spine has a natural kyphosis or forward curvature. However, in cases of bad posture, it is increased more than normal so improving thoracic extension or improving spinal range of motion in extension is helpful to help offset the posture strain of the increased kyphosis. The orthotic devices of the present disclosure effectively stretch ligaments and other soft tissues and gently manipulate the spine to improve spinal intersegmental mobility and the ability to improve range of motion in spinal extension by improving the lordosis of the spine. The orthotic devices of the present disclosure also promote fluid and cellular exchange in and around the intervertebral discs. When properly applied to the spine by a person laying supine over the orthotic devices of the present disclosure, the orthotic devices supply a specific force forwardly in the sagittal plane to apply an angular/circular traction to the specified region of the spine, e.g., the cervical spine, the thoracic spine, and/or the lumbar spine of a person.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. An orthotic device for a cervical spine and an upper thoracic spine of a person, the orthotic device comprising:
a base portion having a first base end and a second base end;
a first protruding portion defining a first support surface configured to support the cervical spine at a first region, the first protruding portion spaced a first distance from the base portion;
a second protruding portion defining a second support surface configured to support the cervical spine at a second region, the second protruding portion spaced a second distance from the base portion, wherein the second distance is less than the first distance; and
a third protruding portion defining a third support surface configured to support the upper thoracic spine at a third region, the third protruding portion spaced a third distance from the base portion, wherein the third distance is less than the second distance.

2. The orthotic device of claim 1, wherein the orthotic device tapers from the first base end to the second base end.

3. The orthotic device of claim 1, wherein the first support surface is a first convex surface.

4. The orthotic device of claim 3, wherein the second support surface is a second convex surface.

5. The orthotic device of claim 4, wherein the third support surface is a third convex surface.

6. The orthotic device of claim 5, further comprising a fourth support surface between the first protruding portion and the second protruding portion.

7. The orthotic device of claim 6, further comprising a fifth support surface between the second protruding portion and the third protruding portion.

8. The orthotic device of claim 7, wherein the fourth support surface is a first concave surface.

9. The orthotic device of claim 8, wherein the fifth support surface is a second concave surface.

10. The orthotic device of claim 1, wherein the first protruding portion is located at the first base end.

11. The orthotic device of claim 10, wherein the third protruding portion is located at the second base end.

12. The orthotic device of claim 11, further comprising a ramp portion spanning the second base end and the third protruding portion.

13. The orthotic device of claim 11, wherein the second protruding portion is located between the first protruding portion and the third protruding portion.

14. The orthotic device of claim 1, wherein the second region is spaced from the first region.

15. An orthotic device for a thoracic spine of a person, the orthotic device comprising:
a base portion having a first base end and a second base end;
a first protruding portion defining a first support surface configured to support the thoracic spine at a first region, the first protruding portion spaced a first distance from the base portion; and
a second protruding portion defining a second support surface configured to support the thoracic spine at a second region, the second protruding portion spaced a second distance from the base portion, wherein the second distance is less than the first distance.

16. The orthotic device of claim 15, wherein the first support surface is a first convex surface.

17. The orthotic device of claim 16, wherein the second support surface is a second convex surface.

18. The orthotic device of claim 17, wherein the first protruding portion is located at the second base end.

19. The orthotic device of claim 18, wherein the second protruding portion is located at the first base end.

20. The orthotic device of claim 19, further comprising a first ramp portion spanning the second base end and the first protruding portion.

21. The orthotic device of claim 20, wherein the first ramp portion extends upwards from the second base end at an angle of 45 degrees.

22. The orthotic device of claim 20, further comprising a second ramp portion spanning the first base end and the second protruding portion.

23. The orthotic device of claim 22, wherein the second ramp portion extends upwards from the first base end at an angle of 70 degrees.

24. An orthotic device for a lumbar spine of a person, the orthotic device comprising:
a base portion having a first base end and a second base end;
a first protruding portion defining a first support surface configured to support the lumbar spine at a first region, the first protruding portion spaced a first distance from the base portion;
a second protruding portion defining a second support surface configured to support the lumbar spine at a second region, the second protruding portion spaced a second distance from the base portion, wherein the second distance is less than the first distance; and
a third protruding portion defining a third support surface configured to support the lumbar spine at a third region, the third protruding portion spaced a third distance from the base portion, wherein the third distance is less than the first distance,
wherein the first protruding portion is located between the second protruding portion and the third protruding portion.

25. The orthotic device of claim 24, wherein the first support surface is a first convex surface.

26. The orthotic device of claim 25, wherein the second support surface is a second convex surface.

27. The orthotic device of claim 26, wherein the third support surface is a third convex surface.

28. The orthotic device of claim 24, wherein the third protruding portion is located at the first base end.

29. The orthotic device of claim 28, wherein the second protruding portion is located at the second base end.

30. The orthotic device of claim 19, further comprising a first ramp portion spanning the second base end and the second protruding portion.

31. The orthotic device of claim 30, wherein the first ramp portion extends upwards from the second base end at an angle of 60 degrees.

32. The orthotic device of claim 30, further comprising a second ramp portion spanning the first base end and the third protruding portion.

33. The orthotic device of claim 32, wherein the second ramp portion extends upwards from the first base end at an angle of 45 degrees.
